(19) United States
(12) Patent Application Publication
(10) Pub. No.: US 2017/0367978 A1
(43) Pub. Date: Dec. 28, 2017

(54) AQUEOUS TOPICAL DRUG FORMULATION WITH CONTROLLED RELEASE AND INCREASED STABILITY

(71) Applicant: Phosphorex, Inc., Hopkinton, MA (US)

(72) Inventor: Bin Wu, Sharon, MA (US)

(73) Assignee: Phosphorex, Inc., Hopkinton, MA (US)

(21) Appl. No.: 15/616,760

(22) Filed: Jun. 7, 2017

Related U.S. Application Data
(63) Continuation of application No. PCT/US2015/064911, filed on Dec. 10, 2015.
(60) Provisional application No. 62/090,603, filed on Dec. 11, 2014.

(51) Int. Cl. A61K 9/107; A61K 47/32; A61K 47/14; A61K 47/44; A61K 9/00

(52) U.S. Cl. CPC A61K 9/107; A61K 47/44; A61K 9/0014; A61K 47/14; A61K 47/32

(57) ABSTRACT

This invention provides a topical drug formulation and methods for the preparation thereof. This invention further provides the use of said drug formulation in pharmaceutical compositions and methods of treating mammals. This invention also provides the use of said drug formulation in pharmaceutical compositions for dermatological and transdermal applications. More specifically, this invention provides an aqueous-based drug formulation which has increased storage stability and controlled drug release upon application. The present application also describes materials and methods useful in creating such drug formulations.

### BACKGROUND OF THE INVENTION

Drugs that are effective for use in the treatment of dermatological conditions and diseases, and for transdermal applications are often hydrophobic. This property results in the creation of oil-based topical and transdermal drug formulations. These formulations tend to be oily and sticky, which causes them to be messy, uncomfortable, and difficult to apply. A solution to this problem is to create aqueous formulations; however, the hydrophobic drugs are often unstable in an aqueous medium, which can cause a decrease in efficacy and bioavailability. Another common problem with some topical drug formulations is that the hydrophobic drugs used can produce offensive odors, within both oil and water-based formulations, which prevent patients from wanting to apply the cream or ointment to their skin on a regular basis. Furthermore, two prevalent challenges are 1) creating the ability to store the aqueous formulations without degradation of the drug occurring over time, and 2) allowing for drug release at the application site upon application of the formulation by the patient. These issues create a need for a new type of drug formulation for direct dermal or transdermal application in which the efficacy and stability of the drug are preserved, or even enhanced, while maintaining an overall water soluble composition and providing a means for blocking potentially offensive odors. Creating this type of formulation would also increase the shelf life and bioavailability of the drug.

### SUMMARY OF THE INVENTION

This invention provides an aqueous-based, topical or transdermal formulation comprising an pharmaceutically or cosmetically acceptable composition (e.g., an API or a cosmetically acceptable ingredient)surrounded by a hydrophilic matrix.

This invention also provides a process for creating an aqueous-based topical drug formulation, this process comprising:
(1) Dissolving or suspending one or more drugs in an organic solvent
(2) Emulsifying the aforementioned drug solution or suspension in an aqueous solution
   The aqueous solution may optionally comprise a surfactant or emulsifier
(3) Adding an aqueous polymer solution to the emulsion to increase the overall viscosity and surround aforementioned dissolved or suspended drug solution in the organic solvent.

Thus in one aspect, the invention provides an aqueous-based formulation, comprising: (1) an aqueous hydrophilic matrix; and, (2) an oil-in-water emulsion of: (a) an aqueous solution; and, (b) an organic solvent with a composition dissolved or suspended therein, wherein droplets of the oil-in-water emulsion is dispersed in the aqueous hydrophilic matrix, and, wherein the aqueous-based formulation is formulated for topical or transdermal delivery of the composition to a mammal.

In certain embodiments, the composition is a pharmaceutically acceptable compound. For example, the composition can be an active pharmaceutical ingredient (API), such as a dermatological or immunosuppressive drug. In certain embodiments, the API is selected from the group consisting of tacrolimus and coal tar.

In certain embodiments, the composition is a cosmetically acceptable compound.

In certain embodiments, the formulation is a foam, a cream, a lotion, a gel, a gel-cream, an emollient cream, or a shampoo.

In certain embodiments, the formulation optionally comprises an antimicrobial agent.

In certain embodiments, the composition is stably dissolved or suspended in the organic solvent, and/or does not diffuse to the aqueous solution.

In certain embodiments, the composition is controllably released by a shear and/or a compression stress applied to the formulation as the formulation is being applied topically.

In certain embodiments, the aqueous hydrophilic matrix comprises a pharmaceutically acceptable polymer or a cosmetically acceptable polymer dissolved in water. For example, the polymer may increase viscosity of the formulation.

In certain embodiments, the aqueous solution is water.

In certain embodiments, the aqueous solution further comprises a surfactant or an emulsifier.

In certain embodiments, the organic solvent is a biologically compatible or pharmaceutically/cosmetically acceptable solvent.

In certain embodiments, the organic solvent is liquid at ambient temperatures, or forms a liquid upon heating at 30-60° C.

In certain embodiments, the organic solvent has a viscosity at ambient temperature of about 0.1-10,000 cp, preferably 1-5,000 cp, and more preferably 1-500 cp. For example, the organic solvent may be selected from the group consisting of: triacetin, diacetin, tocopherol, mineral oils, and capryol 90. In certain embodiments, the organic solvent is capryol 90.

In certain embodiments, the composition is hydrophobic.

In certain embodiments, the composition is unstable in the aqueous solution.

In certain embodiments, the composition has an offensive or unpleasant odor.

In certain embodiments, the formulation masks the offensive or unpleasant odor produced by the composition.

In certain embodiments, the composition is substantially insoluble in the organic solvent, but is stably suspended in the organic solvent as nanoparticles (e.g., those with an average size of about 500 nm, 400 nm, 300 nm, 200 nm, 100 nm or less) or microparticles (e.g., those with an average size of about 1-10 μm, or about 2-5 μm).

In certain embodiments, the formulation is used for transdermal or topical applications.

Another aspect of the invention provides a method for preparing an aqueous-based formulation, the method comprising: (1) carrying out (a)-(c) in any desired order: (a) dissolving or suspending a composition (e.g., coal tar or tacrolimus) in an organic solvent (e.g., capryol 90) to form an organic solvent solution or suspension; (b) dissolving an emulsifier (e.g., gelatin) in a first aqueous solution to form an emulsifier solution; and, (c) dissolving a polymer (e.g., polyvinylpyrrolidone) in a second aqueous solution to form a polymer solution; (2) emulsifying the organic solvent solution or suspension with the emulsifier solution to form an oil-in-water emulsion; (3) mixing the polymer solution with the emulsion to form the aqueous-based formulation, wherein droplets of the oil-in-water emulsion is dispersed in the polymer solution, and, wherein the aqueous-base formulation is formulated for topical or transdermal delivery of the composition to a mammal.

In certain embodiments, the first aqueous solution is the same as the second aqueous solution (e.g., both are water).

In certain embodiments, the composition dissolved or dispersed in the organic solvent has a weight fraction of the composition in the polymeric solution of between 0.01-90 wt %, preferably between 0.1-50 wt %.

In certain embodiments, the organic solvent solution or suspension is added to the emulsifier solution at about 50° C. with stirring at about 300 rpm for about 2 minutes, before the polymer solution is added and mixed for an additional 3 minutes.

Another aspect of the invention provides a method for preparing an aqueous-based formulation, the method comprising: (1) carrying out (a) & (b) in any desired order: (a) dissolving or suspending a composition in an organic solvent to form an organic solvent solution or suspension; and, (b) dissolving an emulsifier and/or a polymer in an aqueous solution to form an emulsifier solution; (2) emulsifying the organic solvent solution or suspension with the emulsifier/polymer solution to form the aqueous-based formulation, wherein the aqueous-based formulation is formulated for topical or transdermal delivery of the composition to a mammal.

In certain embodiments, the composition dissolved or dispersed in the organic solvent has a weight fraction of the composition in the emulsifier/polymer solution of between 0.01-90 wt %, preferably between 0.1-50 wt %.

In certain embodiments, the emulsifier and the polymer are different.

Yet another aspect of the invention provides a method of treating a disease or condition, comprising topically applying any of the subject aqueous-based formulation to the skin of a mammalian patient.

In certain embodiments, the method further comprises controllably releasing the composition by applying a shear and/or a compression stress to the formulation as the formulation is being applied topically.

It should be understood that any embodiments described herein, including those only described under one but not other aspects of the invention, and those only described in the examples, are intended to be able to combine with any one or more other embodiments, unless explicitly disclaimed.

### DETAILED DESCRIPTION OF THE INVENTION

### A. Embodiments of the Invention

The present invention provides an aqueous-based topical formulation comprising an emulsion of an organic solvent containing a composition(s) that does not diffuse out when emulsified with an aqueous solution, mixed with an aqueous polymer solution that increases the overall viscosity and surrounds the drug(s) contained within the organic solvent.

In the present drug formulation, the organic solvent solution may comprise from 0.1% to 95% by volume of the above mentioned composition, preferably from 5% to 50% by volume.

The organic solvent used in the drug formulation described herein is preferably a biologically compatible solvent. Several different kinds of biologically compatible solvents may be used in creating the present invention.

In the following description it will be understood that the nature of the solvents is not critical beyond those particular qualifications set forth below, and may generally be any such known materials conventionally employed and which are accepted in the food and pharmaceutical industry.

The solvent, or mixtures thereof, may be liquid at room temperature, although in some cases, mild heating of a solid solvent to form a liquid is acceptable. A biologically compatible solvent capable of solubilizing the therapeutically or cosmetically active compound may be used.

The biologically compatible solvent may have a viscosity at ambient temperature ranging from 0.1 to 10,000 centipoise, preferably 1 to 5,000 centipoise, more preferably from 1 to about 500 centipoise. For example, triacetin, diacetin, tocopherol, capryol 90, silicone oil, or mineral oils may be used.

Ideally, the biologically compatible solvent is not miscible, or is only partially miscible with water.

Other examples of biologically compatible solvents that may be used include such oils listed in U.S. Pat. No. 5,633,266, the disclosure of which is hereby incorporated in its entirety by reference herein and include CAPTEX™ 200, WHITEPSOL™H-15, and MYVACET™9-45K, hydrogenated cocoa oil, coconut oil, elm seed oil, palm oil, cottonseed oil, soybean oil, parsley seed oil, mustard seed oil, linseed oil, tung oil, pomegranate seed oil, laurel oil, rapeseed oil, corn oil, evening primrose oil, maize oil, olive oil, persic oil, poppy seed oil, safflower oil, sesame oil, soya oil, sunflower oil, ethyl oleate oil, Japanese anise oil, oil of eucalyptus, rose oil, almond oil, arachis oil, castor oil, mineral oil, peanut oil, vegetable oil and derivatives, sucrose polyester, silicone oil, and paraffin oil.

The biologically compatible solvent may also be those that are solid or semi-solid at ambient temperatures but melt into liquid when heated, such as lipids. Examples of useful lipids are phospholipids, saturated and unsaturated fatty acids, and lysolipids. For example, dipalmitoylphosphatidylcholine (DPPC) or distearoylphosphatidylcholine (DSPC) may be used. Other examples of lipids that may be used include, but are not limited to, dioleoylphosphatidylcholine, dimyristoylphosphatidylcholine, dipentademayoylphosphatidlycholine, dilauroylphosphatidylcholine, dioleoylphosphatidylcholine, phosphatidylethanolamines (e.g., dioleoylphosphatidylethanolamine), phosphatidylserine, phos sphatidylglycerol, phosphatidylinositol, sphingolipids (e.g., sphingomyelin), glycolipids (e.g., ganglioside GM1 and GM2), glucolipids, sulfatides, glycosphingolipids, phosphatidic acid, lipids bearing polymers (e.g., bearing polyethylenegylcol (“PEGylated lipids”), chitin, hyaluronic acid or polyvinylpyrrolidone), lipids bearing polysaccharides (e.g., bearing sulfonated mono-, di-, oligo- or polysaccharides), cholesterol, cholesterol sulfate, cholesterol hemusuccinate, tocopheral hemisuccinate, lipids with ether and ester-linked fatty acids, polymerized lipids, diacetyl phosphate, stearylamine, cardiolipin, phospholipids with short chain fatty acids of 6-8 carbons in length, synthetic phospholipids with asymmetric acyl chains (e.g., with one acyl chain of 6 carbons and another acyl chain of 12 carbons), 6-(5-cholesten-3-β-yloxy)-thio-β-D-galactopyranoside, digalactosyldiglyceride, 6-(5-cholesten-3-β-yloxy)hexyl-6-amino-6-deoxy-1-thio-β-D-galactopyranoside, 6-(5-cholesten-3-β-yloxy)hexyl-6-amino-6-deoxyl-1-thio-.alpha.-D-mannopyranoside, 12-(((7′-diethylaminocoumarin-3-yl) carbonyl)methylamino)-octademayoic acid, N-[12-(((7′-diethylaminocoumarin-3-yl) carbonyl)methyl-amino)octadem-ayoyl]-2-aminopalmitic acid, cholesteryl(4′trimethylammonio)butanoate, N- succinyldioleoylphosphatidylethanolamine, 1,2-dioleoyl-sn-glycerol, 1,2,-pipalmitoyl-sn-3-succinylgglycerol, 1,3-dipalmitoyl-2-sucinylglycerol, 1-hexadecyl-3-palmitoylglycerophosphoethanolamine, palmitoylhomocysteine, and combinations thereof.

Additionally, PEGylated lipids that may be used include, but are not limited to, poly(ethylene glycol) (PEG)-based lipids having a molecular weight of between about 1,000 Daltons and 10,000 Daltons, for example about 2,000 Daltons, or 8,000 Daltons.

Saturated and unsaturated fatty acids that may be used include molecules that contain between 12 carbon atoms and 22 carbon atoms in either linear or branched form. Some examples of specific saturated fatty acids that may be used include, but are not limited to, lauric, myristic, palmitic, and stearic acids. Some examples of specific unsaturated fatty acids that may be used include, but are not limited to, lauroleic, physeteric, myristoleic, palmitoleic, petroselinic, linoleic, and oleic acids. Some examples of specific branched fatty acids that may be used include, but are not limited to, isolauric, isomyristic, isopalmitic, and isostearic acids and isoprenoids.

A variety of therapeutically active compounds may be formulated into topical, dermatological, or transdermal formulations using the present invention. The specific nature of a therapeutically active compound to be used may be determined by the kind of disease or disorder that is intended to be treated. For example, immunosuppressive agents may be used as therapeutically active compounds, e.g., tacrolimus, among other kinds of drugs.

Other therapeutically active compounds that may be used in the present invention include, but are not limited to:

analgesics/antipyretics (e.g., aspirin, acetaminophen, ibuprofen, naproxen sodium, buprenorphine hydrochloride, propoxyphene hydrochloride, propoxyphene napsylate, meperidine hydrochloride, hdyromorphone hydrochloride, morphine sulfate, oxycodone hydrochloride, codeine phosphate, dihydrocodeine bitartrate, pentazocine hydrochloride, hydrocodone bitartrate, levorphanol tartrate, diflunisal, trolamine salicylate, nalbuphine hydrochloride, mefenamic acid, butorphanol tartrate, choline salicylate, butalbital, phenyltoloxamine citrate, diphenhydramine citrate, methotrimeprazine, cinnamedrine hydrochloride, meprobamate, and the like);

anesthetics (e.g., halothane, isoflurane, methoxyflurane, propofol, thiobarbiturates, xenon and the like); antiasthmatics (e.g., Azelastine, Ketotifen, Traxanox, and the like);

antibiotics (e.g., neomycin, streptomycin, chloramphenicol, cephalosporin, ampicillin, penicillin, tetracycline, and the like);

antidepressants (e.g., nefopam, oxypertine, doxepin hydrochloride, amoxapine, trazodone hydrochloride, amitriptyline hydrochloride, maprotiliine hydrochloride, phenelzine sulfate, desipramine hydrochloride, nortriptyline hydrochloride, tranylcypromine sulfate, fluoxetine hydrochloride, doxepin hydrochloride, imipramine hydrochloride, isocarboxazid, desipramine hydrochloride, trimipramine maleate, protriptyline hydrochloride, and the like);

antidiabetics (e.g. biguanides, hormones, sulfonylurea derivatives, and the like); antifungal agents (e.g., griseofulvin, ketoconazole, amphotericin B, Nystatin, candicidin and the like);

antihypertensive agents (e.g., propranolol, propafenone, oxyprenolol, nifedipine, reserpine, trimethaphan camsylate, phenoxybenzamine hydrochloride, pargyline hydrochloride, deserpidine diazoxide, guanethidine monosulfate, minoxidil, rescinamine, sodium nitroprusside, rauwolfia serpentina, alseroxylon, phentolamine mesylate, reserpine, and the like);

anti-inflammatories (e.g., (non-steroidal) indomethacin, naproxen, ibuprofen, ramifenazone, piroxicam, (steroidal) cortisone, dexamethasone, fluazacort, hydrocortisone, prednisolone, prednisone, and the like);

antineoplastics (e.g., Adriamycin, cyclophosphamide, actinomycin, bleomycin, duanorubicin, doxorubicin, epirubicin, mitomycin, methotrexate, flurorouracil, carboplatin, carmustine (BCNU), methyl-CCNU, cisplatin etoposide, interferons, camptothcin and derivatives thereof, phenesterine, taxol and derivatives thereof, vinblastine, vincristine, tamoxifen, etoposide, piposulfan, and the like);

antianxiety agents (e.g., lorazepam, buspirone, hydrochloride, prazepam, chlordiazepoxide hydrochloride, oxazepam, clorazepate dipotassium, diazepam, hydroxyzine pamoate, hydroxyzine hydrochlorie, alprazolam, droperidol, halazepam, chlorlene, and the lmezanone, dantrolene, and the like);

antimigraine agents (e.g., ergotamine tartrate, propranolol, hydrochloride, isometheptene mucate, dichloralphenazone, and the like);

sedatives/hypnotics (e.g., barbiturates (e.g., pentobarbital, pentobarbital sodium, secobarbital sodium), benzodiazepines (e.g., flurazepam hydrochloride, triazolam, tomazeparm, midazolam hydrochloride, and the like);

anti-anginal agents (e.g., beta-adrenergic blockers, calcium channel blockers (e.g., nifedipine, diltiazem hydrochloride, and the like), nitrates (e.g., nitroglycerin, isosorbide dinitrate, pentaerythritol tetranitrate, erythrityl tetranitrate, and the like));

antipsychotic agents (e.g., haloperidol, loxapine succinate, loxapine hydrochloride, thioridazine, thioridazine hydrochloride, thiothixene, fluphenazine enanthate, trifluoperazine hydrochloride, chlorpromazine hydrochloride, perphanazine, lithium citrate, prochlorperazine, and the like);

antimanic agents (e.g., lithium carbonate);

antiarrythmics (e.g., amiodarone, related derivatives of amiodarone, bretylium tosylate, esmolol hydrochloride, verapamil hydrochloride, encainide hydrochloride, digoxin, digitoxin, mexiletine hydrochloride, disopyramide phosphate, procainamide hydrochloride, quinidine sulfate, quinidine gluconate, quinidine polygalacturonate, flecainide acetate, tocainide hydrochloride, lidocaine hydrochloride, and the like);

antiarthritic agents (e.g., phenylbutazone, sulindac, penicillamine, salsalate, piroxicam, azathioprine, indomethacin, meclofenamate sodium, gold sodium thiomalate, ketoprofen, auranofin, aurothioglucose, tolmetin soium, and the like);

anti-gout agents (e.g., colchicine, allopurinol, and the like);

anticoagulants (e.g., heparin, heparin sodium, warfarin sodium, and the like);

thrombolytic agents (e.g., urokinase, streptokinase, altoplase, and the like);

antifibrinolytic agents (e.g. aminocaproic acid);

hemorheologic agents (e.g., pentoxifylline);

antiplatelet agents (e.g., aspirin, phenobarbital empiriri, ascription, and the like);

anticonvulsants (e.g., valproic acid, divalproate sodium, phenytoin, phenytoin sodium, clonazepam, primidone, phenobarbital, phenobarbital sodium, carbamazepine, amobarbital sodium, methsuximide, metharbital, mephobarbital, mephenytoin, phensuximide, paramethadione, ethotoin, phenacemide, secobarbital sodium, clarazepate dipotassium, trimethadione, and the like);

anti-Parkinson agents (e.g., ethosuximide, and the like);

antihistamines/antipruritics (e.g., hydroxyzine hydrochloride, diphenhydramine hydrochloride, chlorpheniramine maleate, brompheniramine maleate, cyproheptadine hydrochloride, terfenadine, clemastine fumarate, triprolidine hydrochloride, carbinoxamine maleate, diphenylpyraline hydrochloride, phenindamine tartrate, azatadine maleate, tripelennamine hydrochloride, dexchloropheniramine maleate, methdilazine hydrochloride, trimeprazine tartrate and the like);

agents useful for calcium regulation (e.g., calcitonin, parathyroid hormone, and the like);

antibacterial agents (e.g., amikacin sulfate, aztreonam, chloramphenicol, chloramphenicol palmitate, chloramphenicol sodium succinate, ciprofloxacin hydrochloride, clindamycin hydrochloride, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethane sodium, colistin sulfate, and the like);

antiviral agents (e.g., interferon gamma, zidovudine, amantadine hydrochloride, ribavirin, acyclovir, and the like);

antimicrobials (e.g., cephalosporins (e.g., cefazolin sodium, cephradine, cefaclor, cephapirin sodium, ceffizoxime sodium, cefoperazone sodium, cefotetan disodium, cefutoxime azotil, cefotaxime sodium, cefadroxil monohydrate, ceftazidime, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, cefuroxime sodium, and the like), prythronyclinecins, penicillins (e.g., ampicillin, amoxicillin, penicillin G benzathine, cyclacillin, ampicillin sodium, penicillin G potassium, penicillin V potassium, piperacillin sodium oxacillin sodium, bacampicillin hydrochloride, cloxacillin sodium, ticcarcillin disodium, azlocillin sodium, carbenicillin indanyl sodium, penicillin G procaine, methicillin, sodium, nafcillin sodium, and the like), tetracyclines (e.g., tetracycline hydrochloride, doxycycline hyclate, minocycline hydrochloride, and the like), and the like);

anti-infectives (e.g., GM-CSF);

bronchodilators (e.g., sympathomimetics (e.g., sympathomimetics (e.g., epinephrine hydrochloride, metaproterenol sulfate, terbutaline sulfate, isoetharine, isoetharine mesylate, isoetharine hydrochloride, albuterol sulfate, albuterol, bitolterol, mesylate isoproterenol hydrochloride, terbutaline sulfate, epinephrine bitartrate, metaproterenol sulfate, epinephrine), anticholinergic agents (e.g., ipratropium bromide), xanthines (e.g., aminophylline, dyphilline, metaproterenol sulfate, aminophylline), mast cell stabilizers (e.g., cromolyn sodium), inhalant corticosteroids (e.g., flurisolidebeclomethasone dipropionate, beclomethasone dipropionate (BDP), ipratropium bromide, budesonide, ketotifen, salmeterol, xinafoate, terbutaline sulfate, triamcinolone, theophylline, nedocromil sodium, metaproterenol sulone cypionatefate, albuterol, flunisolide, and the like);

dermatological drugs (e.g., retapamulin, alefacept, Avita® gel, Bactroban® cream, erythromycin 3%-benzoyl peroxide-5%, botulinum toxin type A, cefazolin and dextrose USP, chlorhexidine gluconate, clindamycin phosphate, pokofilox, dalbavancin, deramgraft-TC, desonide, adapalene gel, dynabac, elidel, vismodegib, norethindrone acetate and ethinyl estradiol, ketoconazole, azelaic acid, finevin, firazyr, gralise, Horizant®, iamin, Invanz®, iontocaine, IvyBlock®, efinaconazole, tavaborole, sodium sulfacet amide, terbinafine hydrochloride, azficel-T, enoxaparin sodium, lustra, betamethasone valerate, luliconazole, trametinib, 1% butenafine hydrochloric acid, MetroLotion®, minoxidil, brimonidine, noritate, omnicef, norgestimate/ethinyl estradiol, ingenol mebutate, propecia, tacrolimus, becaplermin, tretinoin, salagen, acyclovir, tedizolid phosphate, SERPACWA, ivermectin, ustekinumab, sulfamylon, peginterferon alfa-2b, dabrafenib, tazorac, ceftaroline fosamil, thalomid, tigecycline, mechlorethamine, polidocanol, clindamycin phosphate and tretinoin, desonide, telavancin, levocetirizine dihydrochloride, ipilimumab, vemurafenib, imiquimod, coal tar and the like;

hormones (e.g., androgens (e.g., danazol, testosterone cypionate, fluoxymesterone, ethyltostosterone, testoreone enanihate, methyltestosterone, fluoxymesterone, testosterone cypionate), estrogens (e.g., estriadiol, estropipate, conjugated estrogens), progestins (e.g., methoxyprogesterone acetate, norethindrone acetate), corticosteroids (e.g., triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate, methylprednisolone sodium succinate, hydrocortisone sodium succinate, triamcinolone hexacatonide, hydrocortisone, hydrocortisone cypionate, prednisolone, fluorocortisone acetate, paramethasone acetate, prednisolone tebulate, prednisolone, acetate, prednisolone sodium phosphate, hydrocortisone sodium succinate, and the like), thyroid hormones (e.g., levothyroxine sodium) and the like), and the like;

hypoglycemic agents (e.g., human insulin, purified beef insulin, purified pork insulin, glyburide, chlorpropamide, glipizide, tolbutamide, tolazaminde, and the like);

hypolipidemic agents (e.g., clofibrate, dextrothyroxine sodium, probucol, lovastatin, niacin, and the like);

proteins (e.g., deoxyribonuclease, alginase, superoxide dismutase, lipase, and the like);

nucleic acids (e.g., sense or anti-sense nucleic acids encoding any therapeutically useful protein, including any of the proteins described herein, and the like);

agents useful for erythropoiesis stimulation (e.g., erythropoietin);

antiulcer/antireflux agents (e.g., famotidine, cimetidine, ranitidine hydrochloride, and the like);

antinauseants/antiemetics (e.g., meclizine hydrochloride, nabilone, prochlorperazine, dimenhydrinate, promethazine hydrochloride, thiethylperazine, scopolamine, and the like);

oil-soluble vitamins (e.g., vitamins A, D, E, K, and the like);

as well as other drugs such as mitotane, visadine, halonitrosoureas, anthrocyclines, ellipticine, and the like.

Other examples of the therapeutic compounds that may be formulated in the present invention for use in topical, dermatological, or transdermal formulations are those active ingredients in traditional Chinese medicine, for example, cucurmin and indirubin.

Although the technology described in the current invention is particularly useful for creating aqueous-based topical drug formulations for hydrophobic drugs, it can also be used for delivery of hydrophilic drugs for other benefits such as improvement in drug stability, pharmo-dynamics and pharmo-kinetics. Examples of hydrophilic drugs are small molecule drugs, proteins, peptides, antibodies, oligonucleotides, vaccines, and hormones.

As described above, a biocompatible solvent capable of solubilizing the therapeutically active compound may be used in the present invention. In one embodiment of the current invention, at least one therapeutically active compound is dissolved in said biocompatible solvent to form a homogeneous solution. The weight fraction of the drug in the organic solvent solution, which is defined as the total mass of the drug dissolved divided by the total mass of the solvent, is between 0.1%-90%, preferably between 1%-50%.

Alternatively, a biocompatible solvent that does not solubilize the therapeutically active compound may also be used as long as the therapeutically active compound can be made into small-sized solid particles and suspended in said biocompatible solvent to form a stable suspension. The therapeutically active compound can be suspended in the suspending solvent by the use of mechanical forces and surfactants to form a stable drug suspension. In the case the therapeutically active compound is suspended as particles in the biocompatible solvent, it is preferred that the particles are small in size, preferably below 10 microns, more preferably below 2 microns, even more preferably below 500 nm. The weight fraction of the drug in the organic solvent suspension is between 0.1-90 wt %, preferably between 1-50 wt %.

There are various ways of making therapeutically active compounds into small sized particles, for example, by milling, homogenization, or precipitation techniques. Exemplary methods of making nanoparticulate compositions are described in U.S. Pat. No. 5,145,684 for “Surface Modified Drug Nanoparticles.” Methods of making nanoparticulate compositions are also described in U.S. Pat. No. 5,518,187 for “Method of Grinding Pharmaceutical Substances;” U.S. Pat. No. 5,156,842 for “Liquid Suspension for Oral Administration;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat No. 5,510,118 for “Process of Preparing Therapeutic Compositions Containing Nanoparticles;” U.S. Pat. No. 5,470,583 for “Method of Preparing Nanoparticle Compositions Containing Charged Phospholipids to Reduce Aggregation;” and U.S. Pat. No. 7,390,505 for “Nanoparticulate Topiramate Formulations,” all of which are specifically incorporated by reference.

1. Milling to Obtain Nanoparticulate Drug Dispersions

Milling the therapeutically active compound to obtain a nanoparticulate dispersion comprises dispersing therapeutically active compound particles in a liquid dispersion media in which it is poorly soluble, followed by applying mechanical means in the presence of grinding media to reduce the particle size of the therapeutically active compound to the desired effective average particle size. The dispersion media is a biocompatible solvent.

The drug particles can be reduced in size in the presence of at least one surface stabilizer. Alternatively, the drug particles can be contacted with one or more surface stabilizers after attrition. Other compounds, such as a diluent, can be added to the drug/surface stabilizer composition during the size reduction process. Dispersions can be manufactured continuously or in a batch mode.

2. Precipitation to Obtain Nanoparticulate Drug Suspensions

Another method of forming the desired nanoparticulate drug suspension is by microprecipitation. This is a method of preparing stable dispersions of active agents in the presence of one or more surface stabilizers and/or one or more colloid stability enhancing surface active agents. Such a method comprises, for example: (1) dissolving the drug in a first solvent; and (2) mixing the drug solution from step (1) with a second solvent to form the nanoparticles, the first solvent is miscible with the second solvent and the active ingredient is insoluble or poorly soluble in the second solvent and 3) removing the first solvent from the nanoparticle suspension, the removal of the first solvent can be performed by dialysis or diafiltration, followed by concentration of the dispersion by conventional means

3. Homogenization to Obtain Nanoparticulate Drug Suspensions

Exemplary homogenization methods of preparing active agent nanoparticulate compositions are described in U.S. Pat. No. 5,510,118, for “Process of Preparing Therapeutic Compositions Containing Nanoparticles.”

This method comprises dissolving the drug in a solvent, followed by emulsifying the resulting drug solution in a pharmaceutically acceptable oil, and then removing the solvent to form drug particles.

The drug particles can be reduced in size in the presence of at least one surface stabilizer. Alternatively, the drug particles can be contacted with one or more surface stabilizers either before or after attrition. Other compounds, such as a diluent, can be added to the drug/surface stabilizer composition before, during, or after the size reduction process. Dispersions can be manufactured continuously or in a batch mode.

The organic solvent solution in the present invention, i.e. the biocompatible solvent containing therapeutic compounds dissolved or suspended therein, is dispersed in an aqueous or a non-aqueous media to form an emulsion by mechanical forces and optionally by the presence of one or a mixture of emulsifiers. The drug dissolved or dispersed in the biocompatible solvent has a weight fraction of the drug in the polymeric solution of between 0.01-90 wt %, preferably between 0.1-50 wt %.

The polymeric solution in the present invention comprises a polymeric material and optionally an emulsifier. Said polymeric material may be a pharmaceutically acceptable polymer. Combinations of more than one polymer can be used in the invention. Useful polymers which can be employed in the invention include, but are not limited to, known natural or synthetic pharmaceutical excipients. Such excipients include various polymers, low molecular weight oligomers, and natural products. In the present invention, said polymer is preferably a water-soluble polymer. Polymers having number average molecular weights of from 500 to 50 million, preferably from 2,500 to 5 million, may be used. Polymers that are preferred in the present invention include but are not limited to polyvinyl alcohol, poly(vinyl acetate), polyvinylpyrrolidone, pol(acyrylic acid), poly(acrylic acid) ammonium salt, poly (acrylic acid) sodium salt, polyacrylamide, poly(ethylene oxide), poly(ethylene glycol), poly(hydroxyethyl methacrylate), polyethyleneimine, poly(N-isopropyl acrylamide), starch, cellulose, dextran, gelatin, chitin, chitosan, and the copolymers and mixtures thereof.

Representative examples of other useful polymers include hydroxypropyl methylcellulose, hydroxypropylcellulose, casein, lecithin (phosphatides), gum acacia, cholesterol, tragacanth, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® e.g., Tween 20® and Tween 80® (ICI Specialty Chemicals)), polyethylene glycols (e.g., Carbowaxs 3550° and 934° (Union Carbide)), polyoxyethylene stearates, carboxymethylcellulose calcium, carboxymethylcellulose sodium, methylcellulose, hydroxyethylcellulose, hydroxypropylmethylcellulose phthalate, noncrystalline cellulose, 4-(1,1,3,3-tetramethylbutyl)-phenol polymer with ethylene oxide and formaldehyde (also known as tyloxapol, superione, and triton), polaxamers (e.g., Pluronic F-68® and F108®, which are block copolymers of ethylene oxide and propylene oxide), poloxamines (e.g., Tetronic 908®, also known as Poloxamin 908®, which is a tetrafunctional block copolymer derived from sequential addition of propylene oxide and ethylene oxide to ethylenediamine (BASF

Wyandotte Corporation, Parsippany, N.J.)), Tetronic 1508® (T-1508) (BASF Wyandotte Corporation), Tritons X-200®, which is an alkyl aryl polyether sulfonate (Rohm and Haas), PEG-derivatized phospholipid, PEG-derivatized cholesterol, PEG-derivatized cholesterol derivative, PEG-derivatized vitamin A, PEG-derivatized vitamin E, lysozyme, random copolymers of vinyl pyrrolidone and vinyl acetate, and the like.

As already mentioned, an emulsifier may also be used to assist in the creation of an emulsion. The emulsifier is a pharmaceutically acceptable surfactant, which may be a small molecule, oligomer, or polymer. It may be nonionic, cationic, or anionic. It may be of natural or synthetic origin.

Representative examples of emulsifiers include, but are not limited to, gelatin, casein, lecithin (phosphatides), gum acacia, cholesterol, tragacanth, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens), polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypopylcellulose, hydroxypropylmethylcellulose phthalate, microcrystalline cellulose, magnesium aluminum silicate, triethanolamine, plyvinyl alcohol, polyvinylpyrrolidene (PVP), stearic acid, benzalkonium chloride, calcium stearate, glycerol monostearate, cetostearyl alcohol, cetomacrogol emulsifying wax, and sorbitan esters. Most of these surface modifiers are known pharmaceutical excipients and are described in detail in the Handbook of Pharmaceutical Excipients, published jointly by the American Pharmaceutical Association and The Pharmaceutical Society of Great Britain, the Pharmaceutical Press, 1986.

Other examples of surfactants include tyloxapol, poloxamers such as Pluronic F-68®, F77®, and F108®, which are block copolymers of ethylene oxide and propylene oxide, and polyxamines such as Tetronic 908® (also known as Poloxamine 908®), which is a tetrafunctional block copolymer derived from sequential addition of propylene oxide and ethylene oxide to ethylenediamine, available from BASF, dextran, lecithin, dialkylesters of sodium sulfosuccinic acid, such as Aerosol OT, which is a dioctyl ester of sodium sulfosuccinic acid, available from American Cyanamid, Duponol P, which is a sodium lauryl sulfate, available from DuPont, Triton X-200, which is an alkyl aryl polyether sulfonate, available from Rohm and Haas, Tween 20® and Tween 80®, which are polyoxyethylene sorbitan fatty acid esters, available from ICI Specialty Chemicals, Carbowax 3550® and 934®, which are polyethylene glycols available from Union Carbide; Crodesta F-1010®, which is a mixture of sucrose stearate and sucrose distearate, available from Croda Inc., CrodestaSL-40®, which is available from Croda, Inc., and SA9OHCO, which is C₁₈H₃₇ -CH₂(CON(CH₃) CH₂(CHOH)₄CH₂OH)₂, decanoyl-N-methylglucamide, n-decylβ-D-glucopyranoside; n-dodecylβ-D-glucopyranoside, n-dodecylβ-D-maltoside, heptanoyl-N-methylglucamide, n-heptyl-β-D-glucopyranoside, n-hypylβ-D-thioglucoside, n-hexylβ-D-glucopyanoside, nonanoyl-N-methylglucamid, n-noylβ-D-glucopyranoside, octanoyl-N-methylglucamide, n-octyl-β-D-glucopyranoside, octyl-β-D-thioglucopyranoside, and the like.

Generally, there are two preferred methods which are used in the present invention to prepare the aqueous-based drug formulation. In both cases, a polar solvent preferably is used to dissolve the chosen polymer. Said polar solvent is exemplified by, but not limited to, water, methyl alcohol, ethyl alcohol, isopropyl alcohol, acetone, ethyl acetate, tetrahydrofuran, and the mixture thereof. Water is highly preferred in the present invention for dissolving the chosen polymer.

The first method used in the present invention to prepare the aqueous-based topical drug formulation comprises the following steps:
1) Creating the organic solvent solution by either dissolving or suspending the drug/composition in the biocompatible solvent, as described above; the drug dissolved or dispersed in the biocompatible solvent has a weight fraction of the drug in the polymeric solution of between 0.01-90 wt %, preferably between 0.1-50 wt %;
2) Dissolving the emulsifier in a polar solvent to form an emulsifier solution. Water is highly preferred as said polar solvent. Heat may be used to facilitate the dissolution of the emulsifier. The concentration of the emulsifier solution is preferably from 0 to 20 wt %, more preferably from 0.001 to 10 wt %.;
3) Dissolving a polymer of choice in the polar solvent described in Step 2) to form a polymeric solution with a concentration of from 0.1 to 99 wt %, preferably from 1 to 50 wt %, more preferably from 5 to 30 wt %;
4) Emulsifying the organic solvent solution prepared in Step 1) with the emulsifier solution prepared in Step 2).
5) Mixing the polymeric solution prepared in Step 3) with the emulsion of Step 4). The volume ratio of the polymeric solution to the emulsion is from 0.01 to 100, preferably 0.1 to 50, more preferably from 1 to 10. After mixing, the stirring speed and temperature are maintained for from 0 to 24 hours, preferably from 5 minutes to 5 hours. The stirring speed is then preferably reduced to 10-5,000 rpm, preferably 50-1,000 rpm. The stirring is continued for 1-240 hours, preferably 5-100 hours. The temperature is preferably maintained at from -10 to 95° C., more preferably from 10 to 50° C.;
6) The drug formulation obtained in Step 5) may be used for topical applications and transdermal applications.

The second method used in the present invention to prepare the topical drug formulation for comprises the following steps:
A. Preparing the drug-containing organic solvent solution by either dissolving or suspending the drug/composition in the biocompatible solvent, as described above; the drug dissolved or dispersed in the biocompatible solvent has a weight fraction of the drug in the polymeric solution of between 0.01-90 wt %, preferably between 0.1-50 wt %;
B. Preparing a solution comprising an emulsifier and a chosen polymer described as above in the polar solvent. The concentration of the emulsifier solution is preferably from 0 to 20 wt %, more preferably from 0.001 to 10 wt %. The concentration of the chosen polymer is from 0.1 to 99 wt %, preferably from 1 to 50 wt %, more preferably from 5 to 30 wt %. Water is the highly preferred solvent in the present invention. It is noted that in this case the emulsifier and the chosen polymer may or may not be the same substance; therefore, the use of either component may be optional;
C. Mixing the organic solvent solution prepared in Step A with the solution prepared in Step B to form an emulsion. The ratio of the organic solvent solution to the polymeric solution is from 0.01 to 100, preferably 0.1 to 50, more preferably from 1 to 10. Heat and mechanical agitation (for example, stirring) may be used to facilitate the formation of the emulsion. The speed of said stirring may be from 10-100,000 rpm, preferably 100-10,000 rpm. A homogenizing device such as a rotor stator, homogenizer or micro-fluidizer can be further used to facilitate the formation of the emulsion.
D. The stirring speed and temperature are maintained from 0 to 24 hours, preferably reduced to 10-5,000 rpm, more preferably 50-1,000 rpm. The stirring is continued for 1-240 hours, preferably 5-100 hours. The temperature is preferably maintained at from −10 to 95° C., more preferably from 10 to 50° C.
E. The drug formulation obtained in Step D may be used for topical applications (i.e., dermatological or transdermal applications).

Optionally, drugs may also be dispersed in the polymeric solution in the present invention.

In certain embodiments of this invention, the drug/composition is coal tar.

In certain other embodiments, the drug/composition is tacrolimus.

### B. Methods for Treatment

The present invention can be used to create a topical formulations of hydrophobic drugs. As mentioned previously, an overall aqueous formulation can provide an increased level of comfort when applied as a cream or ointment, can improve the shelf life of the drug, and can block potentially offensive odors.

The drug containing emulsion is essentially “frozen” within the polymeric solution; therefore, polymer-surrounded drugs will not readily burst without an external applied force. When the patient applies a cream or ointment fabricated using the present invention, applied shear and compressive stresses due to rubbing the formulation against the skin will cause the polymer surrounded drugs to diffuse from the organic solvent solution to the aqueous solution, thus allowing them to dissolve and be released for therapeutic purposes. This allows for maintained drug stability during storage and blocks potentially offensive odors.

In certain embodiments, the present topical formulation is created in the form of foam, lotion, cream, emollient cream, shampoo, or topical solution.

In certain embodiments, additional excipients may be used to create an overall drug/composition formulation. These excipients include, but are not limited to, anhydrous citric acid, cetyl alcohol, cyclomethicone, isopropyl myristate, light mineral oil, polyoxyl 20 cetostearyl ether, potassium citrate monohydrate, propylene glycol, purified water, sorbitan monolaurate, white petrolatum, phenoxyethanol, ethanol (60%), polysorbate 60, potassium citrate, stearyl alcohol, glyceryl monostearate, cetostearyl alcohol, glyceryl stearate, PEG 100 stearate, white wax, chlorocresol, sodium citrate, citric acid monohydrate, cetomacrogol 1000, citric acid, dimethicone 350, imidurea, isopropyl myristate, sodium citrate, alcohol, coco-betaine, polyquaternium-10, sodium laureth sulfate, carbomer 934P, and sodium hydroxide.

The final formulation can comprise an antimicrobial agent.

### EXAMPLES

### Example 1

### Preparation of Topical Coal Tar Formulation with Controlled Release

About 1 gram of gelatin was dissolved in 20 ml of deionized water, about 14 grams of polyvinylpyrrolidone was dissolved in 40 ml of deionized water, and about 1 gram of coal tar was dissolved in 4 ml of Capryol 90. A jacketed reactor was prepared with a water bath at a temperature of 50° C. and a stirring speed of 300 rpm. The gelatin solution was added to the jacketed reactor, then the coal tar/Capryol 90 mixture was added to the gelatin solution in the reactor. Such mixture was stirred for 2 minutes. The polyvinylpyrrolidone solution was added to the aforementioned mixture in the jacketed reactor. The overall mixture was stirred for 3 minutes. The heating element of the reactor was turned off, allowing the mixture to cool

Attorney Docket No.: 122394-00202 to room temperature. The final coal tar formulation with controlled release was then collected.

Example 2 Preparation of Topical Tacrolimus Formulation with Enhanced Stability

About 1 gram of gelatin was dissolved in 20 ml of deionized water, about 14 grams of polyvinylpyrrolidone was dissolved in 40 ml of deionized water, and about 42 milligrams of tacrolimus was dissolved in 4 ml of Capryol 90. A jacketed reactor was prepared with a water bath at a temperature of 50° C. and a stirring speed of 300 rpm. The gelatin solution was added to the jacketed reactor, then the tacrolimus/Capryol 90 mixture was added to the gelatin solution in the reactor. Such mixture was stirred for 2 minutes. The polyvinylpyrrolidone solution was added to the aforementioned mixture in the jacketed reactor. The overall mixture was stirred for 3 minutes. The heating element of the reactor was turned off, allowing the mixture to cool to room temperature. The final tacrolimus formulation was then collected.

1. An aqueous-based formulation, comprising:
(1) an aqueous hydrophilic matrix; and,
(2) an oil-in-water emulsion of:
(a) an aqueous solution; and,
(b) an organic solvent with a composition dissolved or suspended therein,
wherein droplets of the oil-in-water emulsion is dispersed in the aqueous hydrophilic matrix, and, wherein the aqueous-based formulation is formulated for topical or transdermal delivery of the composition to a mammal.

2. The aqueous-based formulation of claim 1, wherein the composition is a pharmaceutically acceptable compound or a cosmetically acceptable compound.

3. The aqueous-based formulation of claim 2, wherein the composition is an active pharmaceutical ingredient (API).

4. The aqueous-based formulation of claim 3, wherein the API is a dermatological or immunosuppressive drug.

5-8. (canceled)

9. The aqueous-based formulation of claim 1, wherein the formulation is a foam, a cream, a lotion, a gel, a gel-cream, an emollient cream, or a shampoo.

10. The aqueous-based formulation of claim 1, wherein the formulation optionally comprises an antimicrobial agent.

11. The aqueous-based formulation of claim 1, wherein the composition is stably dissolved or suspended in the organic solvent, and/or does not diffuse to the aqueous solution.

12. The aqueous-based formulation of claim 1, wherein the composition is controllably released by a shear and/or a compression stress applied to the formulation as the formulation is being applied topically.

13. The aqueous-based formulation of claim 1, wherein the aqueous hydrophilic matrix comprises a pharmaceutically acceptable polymer or a cosmetically acceptable polymer dissolved in water.

14. The aqueous-based formulation of claim 13, wherein the polymer increases viscosity of the formulation.

15. The aqueous-based formulation of claim 1, wherein the aqueous solution is water.

16. The aqueous-based formulation of claim 1, wherein the aqueous solution further comprises a surfactant or an emulsifier.

17. The aqueous-based formulation of claim 1, wherein the organic solvent is a biologically compatible or pharmaceutically/cosmetically acceptable solvent.

18. The aqueous-based formulation of claim 1, wherein the organic solvent is liquid at ambient temperatures, or forms a liquid upon heating at 30-60° C.

19. The aqueous-based formulation of claim 1, wherein the organic solvent has a viscosity at ambient temperature of about 0.1-10,000 cp, preferably 1-5,000 cp, and more preferably 1-500 cp.

20-21. (canceled)

22. The aqueous-based formulation of claim 1, wherein the composition is hydrophobic.

23. The aqueous-based formulation of claim 1, wherein the composition is unstable in the aqueous solution.

24-25. (canceled)

26. The aqueous-based formulation of claim 1, wherein the composition is substantially insoluble in the organic solvent, but is stably suspended in the organic solvent as nanoparticles (e.g., those with an average size of about 500 nm, 400 nm, 300 nm, 200 nm, 100 nm or less) or microparticles (e.g., those with an average size of about 1-10 or about 2-5 μm).

27. (canceled)

28. A method for preparing an aqueous-based formulation, the method comprising:
(1) carrying out (a)-(c) in any desired order:
(a) dissolving or suspending a composition (e.g., coal tar or tacrolimus) in an organic solvent (e.g., capryol 90) to form an organic solvent solution or suspension;
(b) dissolving an emulsifier (e.g., gelatin) in a first aqueous solution to form an emulsifier solution; and,
(c) dissolving a polymer (e.g., polyvinylpyrrolidone) in a second aqueous solution to form a polymer solution;
(2) emulsifying the organic solvent solution or suspension with the emulsifier solution to form an oil-in-water emulsion;
(3) mixing the polymer solution with the emulsion to form the aqueous-based formulation, wherein droplets of the oil-in-water emulsion is dispersed in the polymer solution, and, wherein the aqueous-base formulation is formulated for topical or transdermal delivery of the composition to a mammal.

29-31. (canceled)

32. A method for preparing an aqueous-based formulation, the method comprising:
(1) carrying out (a) & (b) in any desired order:
(a) dissolving or suspending a composition in an organic solvent to form an organic solvent solution or suspension; and,
(b) dissolving an emulsifier and/or a polymer in an aqueous solution to form an emulsifier solution;
(2) emulsifying the organic solvent solution or suspension with the emulsifier/polymer solution to form the aqueous-based formulation,
wherein the aqueous-based formulation is formulated for topical or transdermal delivery of the composition to a mammal.

33-36. (canceled)
